(19) United States
(12) Patent Application Publication
(10) Pub. No.: US 2024/0374882 A1
(43) Pub. Date: Nov. 14, 2024

(54) DEVICE AND METHOD FOR PREPARING MICRONEEDLE

(71) Applicants: SHANGHAI REVEDA MEDICAL BIOTECH CO., LTD., Shanghai (CN); SUZHOU REVEDA MEDICAL BIOTECH CO., LTD., Suzhou, Jiangsu (CN)

(72) Inventors: Binwen CHEN, Shanghai (CN); Ping YAN, Shanghai (CN); Yuan HUANG, Shanghai (CN); Long LIU, Shanghai (CN); Qiuyu QU, Shanghai (CN)

(21) Appl. No.: 18/293,108

(22) PCT Filed: Jul. 30, 2021
(86) PCT No.: PCT/CN2021/109769

§ 371 Date: Jan. 29, 2024

(30) Foreign Application Priority Data
Jul. 28, 2021 (CN) 202110858134.7

(51) Int. Cl. A61M 37/00; B29C 39/24; B29C 39/42; B29C 39/44

(52) U.S. Cl. CPC A61M 37/0015; B29C 39/24; B29C 39/42; B29C 39/44; A61M 2037/0053

(57) ABSTRACT

A device and methods for producing microneedles includes a microneedle female mold, a vacuum chamber, a filling mechanism and an evacuation mechanism. The vacuum chamber includes a feeding cavity, a filling cavity and a discharging cavity, which are independent of one another. The feeding cavity receives the microneedle female mold therein in non-negative pressure state, and the evacuation mechanism evacuates the feeding cavity that has received the microneedle female mold therein. The filling cavity receives, in negative pressure state, the microneedle female mold transported from the feeding cavity in negative pressure state. The discharging cavity receives, in negative pressure state, the microneedle female mold transported from the filling cavity in negative pressure state and to undergo vacuum destruction after the microneedle female mold is received therein. The device and methods more efficiently produces microneedles in large batches and ensuring formation quality of the produced microneedles.

### TECHNICAL FIELD

The present invention relates to the field of microneedle production, in particular, to a device and methods for producing microneedles.

### BACKGROUND

Most therapeutic drugs enter the human body through subcutaneous injection, which is an economic, rapid, direct mode of administration. However, patients themselves cannot use syringes easily, and the pain and fear brought about these devices further limit patient compliance. One of the solutions for this problem is to coat a drug on microneedles (including micron-sized needles) and deliver it transdermally. Transdermal delivery using microneedles allows painless drug delivery and increased patient compliance and safety. Moreover, microneedles can be used to precisely deliver a quantitative amount of a drug to a target site in a desired way. Further, microneedles can be used for skin pretreatment for enhancing skin permeability. Therefore, microneedles are promised with a good clinical application.

A polymer microneedle patch has polymer microneedles capable of piercing the stratum corneum of the human body to form channels, which facilitate drug delivery and transdermal drug absorption. An existing technique for producing polymer microneedles involves: first preparing a polymer solution for forming microneedles; filling the polymer solution into cavities in a microneedle female mold; solidifying the polymer solution in the microneedle female mold; and removing the mold to obtain soluble microneedles. In this technique, the molding is commonly accomplished by embossing using an elastomeric silicone female mold. Specifically, an elastomeric silicone female mold with cavities (of the same shape as the microneedles to be produced) is fabricated in advance, and a prepared polymer solution is coated on a surface of the elastomeric silicone female mold with the cavities. The mold is then placed in a vacuum environment for a period of time to allow the polymer solution to completely fill the cavities in the elastomeric silicone female mold. After that, the mold is transported into a natural environment, and the solution is solidified by some means (e.g., drying or crosslinking), thus forming soluble microneedles.

In the above process, the most critical step is the filling of the polymer solution into the cavities in the elastomeric silicone female mold. Since the cavities in the elastomeric silicone female mold are tiny (some portions thereof may be sized even only a few microns), individually filling them one by one would lack efficiency and impose demanding requirements on the fabrication equipment, creating great challenges in batch production. Simultaneous filling can address batch production, but after the polymer solution is cast onto the surface of the elastomeric silicone female mold, there may be air remaining in the micro-cavities under the solution, which will resist the solution from further movement downward deep into the cavities, eventually making the resulting soluble microneedles inferior in formation quality.

In order to overcome this problem, it has been proposed to pre-treat the empty microneedle female mold with a plasma gas to enhance its surface hydrophilicity and thereby facilitate filling of the polymer solution into the microneedle cavities. However, the increased hydrophilicity of the female microneedle induced by plasma gas pre-treatment diminishes over time. Consequently, depending on a length of time the pre-treated microneedle female mold stays in the system, its hydrophilicity may considerably differ from that of other molds, and may vary across its own surface. This is detrimental to filling of the cavities in the microneedle female mold and can deteriorate consistency of the resulting soluble microneedles. Thus, it is difficult for this approach to ensure formation quality of the produced soluble microneedles.

It has also been proposed to cast the polymer solution onto the surface of the microneedle female mold under negative pressure and then fill the polymer solution into the cavities under the action of the atmospheric pressure. This is reportedly capable of quick, uniform casting over a large-area microneedle mold and fast, high-precision replication of nano-sized microneedles. However, it also fails to address batch production due to low productivity. Moreover, it is faced with the challenge of maintaining a consistent vacuum filling environment, eventually leading to suboptimal formation quality. Further, it lacks of flexibility and cannot accommodate microneedle molds of different sizes. Consequently, frequent equipment disassembly and reassembly is necessary, leading to cumbersome and complicated operation. Apart from these, the conventional techniques are associated with the problems of poor leveling of the solution over the surface of the microneedle female mold, possible contamination of the solution during leveling and insufficient air evacuation from the cavities during solution casting. All of these make the conventional techniques difficult to ensure formation quality of the produced microneedles. Other problems with these techniques for making soluble microneedles include a low level of automation, low productivity and high labor costs.

### SUMMARY OF THE INVENTION

It is an objective of the present invention to provide a device and methods capable of high-throughput production of microneedles in large batches with increased productivity and improved formation quality of the produced microneedles.

To this end, the present invention provides a device for producing microneedles, including a microneedle female mold, a vacuum chamber, a filling mechanism and an evacuation mechanism, the microneedle female mold provided in its surface with grooves matching with the microneedles, the filling mechanism at least partially disposed in the vacuum chamber and configured to release a solution for producing the microneedles, the evacuation mechanism connected to the vacuum chamber and configured to evacuate the vacuum chamber, the vacuum chamber including a feeding cavity, a filling cavity and a discharging cavity, which are independent of one another,
the feeding cavity configured to receive the microneedle female mold under non-negative pressure,
the evacuation mechanism configured to evacuate the feeding cavity that has received the microneedle female mold therein,
the filling cavity configured to receive, under negative pressure, the microneedle female mold transported from the feeding cavity under negative pressure,
the discharging cavity configured to receive, under negative pressure, the microneedle female mold transported from the filling cavity under negative pressure and to undergo vacuum destruction after the microneedle female mold is received therein.

Optionally, the device may further include a controller and vacuum breaker valves communicatively connected to the controller, the vacuum breaker valve including a first vacuum breaker valve and a third vacuum breaker valve, the first vacuum breaker valve disposed in the feeding cavity, the third vacuum breaker valve disposed in the discharging cavity,
the controller configured to open the first vacuum breaker valve to destroy a vacuum in the feeding cavity and to open, after the microneedle female mold in a third state is transported into the discharging cavity under negative pressure, the third vacuum breaker valve to destroy a vacuum in the discharging cavity.

Optionally, the controller may be further communicatively connected to the evacuation mechanism and configured to control the evacuation mechanism to evacuate the discharging cavity, the filling cavity and the feeding cavity.

Optionally, the device may further include a sensor assembly communicatively connected to the controller, the sensor assembly including a first sensor, a second sensor and a third sensor,
wherein the first sensor is disposed in the feeding cavity; the second sensor is disposed in the filling cavity; the third sensor is disposed in the discharging cavity; and the controller is configured to control, based on pressure information detected by the first sensor, the second sensor and the third sensor, degrees of vacuum in the respective cavities.

Optionally, the device may further include a leveling mechanism, wherein the filling cavity defines therein a casting location and a leveling location;
when the filling cavity is under negative pressure and when the microneedle female mold is located at the casting location in the filling cavity, the filling mechanism is configured to cast the solution onto the surface of the microneedle female mold;
after the solution is cast onto the microneedle female mold in the filling cavity, the microneedle female mold is configured to be transported to the leveling location, and the leveling mechanism is configured to perform a leveling process on the microneedle female mold with the solution cast thereon, so that the solution uniformly spreads over the surface of the microneedle female mold.

Optionally, the leveling mechanism may include a picking structure and a driving mechanism, the picking structure including a spindle assembly and a chuck assembly, the driving mechanism including a servo motor and a transmission assembly, the spindle assembly including a spindle and a base, the spindle fixed at the bottom to the base, the chuck assembly including at least three jaws, which are arranged on the base evenly about an axis of the spindle and configured to clamp and retain a tray for carrying the microneedle female mold therein, the servo motor configured to drive the spindle to rotate through the transmission assembly, the axis of the spindle parallel to axes of the cavities.

Optionally, the driving mechanism may further include a pneumatic cylinder assembly, a resilient member and a turntable, the turntable disposed over the spindle so as to be rotatable relative to the spindle, the resilient member coupled to the spindle at one end and to the turntable at the other end,
wherein as a result of the turntable being driven by the pneumatic cylinder assembly to rotate in a first direction, the resilient member stores resilient energy, causing all the jaws to move to their unlocking positions; and
when the pneumatic cylinder assembly stops acting on the turntable, the resilient member releases the stored resilient energy, driving the turntable to rotate in the second direction and causing all the jaws to move to their locking positions.

Optionally, the leveling mechanism may further include at least three press blocks, which are fixed to the base and configured to axially press against the turntable.

Optionally, the chuck assembly may further include guide rails, sliders, fixation bases and stop pins, the guide rails extending radially with respect to the base, the sliders slidably provided on the guide rails, the fixation bases fixed to the sliders, with each of the jaws being secured to a respective one of the fixation bases, the stop pins fixed to the fixation bases, wherein the turntable defines curved stop slots, in which the stop pins are movably inserted, and which have opposite ends spaced from a center of the turntable at different distances;
as a result of the turntable rotating in the first direction, the stop pins move from proximal ends of the curved stop slots to distal ends thereof, thereby causing the jaws to move outwardly, and the stop pins stop upon reaching and buckling the distal ends of the stop slots; and
as a result of the turntable rotating in the second direction, the stop pins move from the distal ends of the curved stop slots to the proximal ends thereof, thereby causing the jaws to move inwardly, and the stop pins stop upon reaching and buckling the proximal ends of the stop slots.

Optionally, the pneumatic cylinder assembly may include a push rod, wherein a fixed stud is provided on the turntable, and the push rod is configured to push the fixed stud and thereby cause the turntable to rotate in the first direction.

Optionally, the device may further include a tray and a conveyor, the tray configured to carry the microneedle female mold therein, the conveyor configured to transport the tray, wherein the leveling mechanism is configured to drive the tray to move and thereby effectuate the leveling process on the microneedle female mold, the movement of the tray including at least one of horizontal rotation, horizontal translation, shaking and vertical swinging.

Optionally, the device may further include a tray for carrying the microneedle female mold therein, wherein the tray is able to carry microneedle female mold of different sizes.

Optionally, the device may further include a loading conveyor, an unloading conveyor and a transfer conveyor, the loading conveyor disposed in a loading zone, the unloading conveyor disposed in an unloading zone,
the loading conveyor configured to transport the tray into the feeding cavity, the unloading conveyor configured to receive the tray from the discharging cavity,
the transfer conveyor configured to receive the empty tray from the unloading conveyor and transport the empty tray to the loading zone.

Optionally, the transfer conveyor may be disposed under the loading conveyor and the unloading conveyor, wherein the device further includes an automated transport device for transporting the empty tray from the unloading conveyor to the transfer conveyor and from the transfer conveyor to the loading conveyor.

Optionally, the device may further include an automated feeding mechanism and an automated loading mechanism both disposed in the loading zone, the automated feeding mechanism configured to transport the microneedle female mold to a loading location, the automated loading mechanism configured to pick up the microneedle female mold from the loading location and place it into the tray on the loading conveyor.

Optionally, the device may further include an automated unloading mechanism and a transfer tray both disposed in the unloading zone, the automated unloading mechanism configured to pick up the microneedle female mold from the tray on the unloading conveyor and place it into the transfer tray.

Optionally, the feeding cavity, the filling cavity and the discharging cavity may be sequentially adjacent, wherein a first hatch is provided at an entrance of the feeding cavity; a common second hatch is provided at both an exit of the feeding cavity and an entrance of the filling cavity; a common third hatch is provided at both an exit of the filling cavity and an entrance of the discharging cavity; and a fourth hatch is provided at an exit of the discharging cavity.

Optionally, the device may further include a first conveyor, a second conveyor and a third conveyor, the first conveyor disposed in the feeding cavity, the second conveyor disposed in the filling cavity, the third conveyor disposed in the discharging cavity.

Optionally, the filling mechanism may be configured to cast the solution onto the microneedle female mold in the filling cavity under negative pressure.

To the above end, the present invention also provides a method for producing microneedles, including:
providing a microneedle female mold provided in its surface with grooves matching with to the microneedles;
placing the microneedle female mold into a feeding cavity under non-negative pressure and then drawing a vacuum in the feeding cavity by an evacuation mechanism so that the feeding cavity and the microneedle female mold are both under negative pressure;
transporting the microneedle female mold under negative pressure from the feeding cavity under negative pressure into a filling cavity under negative pressure, casting a solution for producing the microneedles, under negative pressure, onto the surface of the microneedle female mold by a filling mechanism and performing a leveling process on the microneedle female mold with the solution cast thereon by a leveling mechanism so that the solution is uniformly flattened on the surface of the microneedle female mold;
transporting the microneedle female mold with the solution casted thereon from the filling cavity under negative pressure into a discharging cavity under negative pressure and maintaining the filling cavity under negative pressure;
after the microneedle female mold with the solution casted thereon is transported into the discharging cavity under negative pressure, destroying a vacuum in the discharging cavity under negative pressure so that the discharging cavity is under non-negative pressure, and taking out the microneedle female mold with the solution thereon; and
after the solution on the microneedle female mold solidifies, removing the mold, thereby obtaining the microneedles.

Optionally, the method may further include:
after the microneedle female mold under negative pressure is transported from the feeding cavity under negative pressure into the filling cavity under negative pressure, destroying a vacuum in the feeding cavity under negative pressure so that the feeding cavity is under non-negative pressure and gets ready for reception of the next microneedle female mold therein.

Optionally, the method may further include:
after the vacuum in the discharging cavity under negative pressure is destroyed, transporting the microneedle female mold with the solution thereon out of the discharging cavity under non-negative pressure, thereby emptying the discharging cavity; and
after the discharging cavity under non-negative pressure is emptied, drawing a vacuum in the discharging cavity by the evacuation mechanism so that the discharging cavity is under negative pressure and ready for reception of the next microneedle female mold that has been undergone a leveling process.

Optionally, the method may further include:
at a casting location in the filling cavity, casting the solution onto the surface of the microneedle female mold under negative pressure by the filling mechanism; and
at a leveling location in the filling cavity, performing the leveling process on the microneedle female mold with the solution cast thereon by the leveling mechanism.

Optionally, at the leveling location, the microneedle female mold may be carried in a tray, wherein the leveling mechanism drives the tray to move and thereby effectuates the leveling process on the microneedle female mold, the movement of the tray including at least one of horizontal rotation, horizontal translation, shaking and vertical swinging.

Optionally, the method may include: passing the microneedle female mold in a tray successively through the feeding cavity, the filling cavity and the discharging cavity, wherein microneedle female mold of different sizes can be transported in the tray.

Optionally, the method may include:
transporting the tray into the feeding cavity on a loading conveyor in the loading zone and receiving the tray from the discharging cavity onto an unloading conveyor in an unloading zone; and
receiving the empty tray from the unloading conveyor onto a transfer conveyor and transporting the empty tray on the transfer conveyor into the loading zone.

Optionally, the transfer conveyor may be arranged under the loading conveyor and the unloading conveyor, wherein the method further includes:
transporting the empty tray from the unloading conveyor onto the transfer conveyor by an automated transport device and transporting the empty tray from the transfer conveyor onto the loading conveyor by the automated transport device.

Optionally, the method may include:
transporting the microneedle female mold to a loading location by an automated feeding mechanism in the loading zone; and
picking up the microneedle female mold from the loading location and placing it into the tray on the loading conveyor by an automated loading mechanism in the loading zone.

Optionally, the method may include:
picking up the microneedle female mold from the tray on the unloading conveyor and placing it into the transfer tray in the unloading zone by an automated unloading mechanism in the unloading zone.

Optionally, a controller may control the evacuation mechanism to evacuate the feeding cavity, the filling cavity and the discharging cavity.

Optionally, in the leveling process on the microneedle female mold, the leveling mechanism may rotate successively at an increasing speed, a constant speed and a decreasing speed.

To the above end, the present invention also provides a method for producing microneedles, including:
providing a microneedle female mold and a vacuum chamber, the microneedle female mold provided in its surface with grooves matching with the microneedles, the vacuum chamber including a filling cavity and a discharging cavity, which are independent of each other;
placing the microneedle female mold into the filling cavity under non-negative pressure and casting a solution for producing the microneedles onto the surface of the microneedle female mold by a filling mechanism;
performing a leveling process on the microneedle female mold by a leveling mechanism so that the solution is uniformly flattened on the surface of the microneedle female mold;
drawing a vacuum in the filling cavity by an evacuation mechanism so that the filling cavity and the microneedle female mold are both under negative pressure;
transporting the microneedle female mold having undergone the leveling process into the discharging cavity under negative pressure, destroying a vacuum in the discharging cavity under negative pressure so that the discharging cavity is under non-negative pressure, and taking out the microneedle female mold with the solution thereon; and
after the solution on the microneedle female mold solidifies, removing the mold.

Optionally, the method may include: passing the microneedle female mold through the filling cavity and the discharging cavity in a tray, wherein at a leveling location, the leveling mechanism drives the tray to move and thereby effectuates the leveling process on the microneedle female mold, the movement of the tray including at least one of horizontal rotation, horizontal translation, shaking and vertical swinging.

In the above device and methods, the independent feeding, filling and discharging cavities decouple vacuum suction, vacuum filling of the microneedle female mold and vacuum destruction operations from one another. This can greatly reduce waiting times during vacuum filling, enhancing overall vacuum filling efficiency. Moreover, prior to vacuum filling, the microneedle female mold has been subjected to vacuum suction for a long period of time, and air in the cavities in the surface of the microneedle female mold has been substantially removed, allowing the solution to fill up the cavities in a desirable way. This not only can effectively ensure formation quality of the resulting microneedles, but can also significantly speed up production and increase the apparatus' throughput, allowing production of microneedles in large batches.

In the above device and methods, the microneedle female mold is subject to vacuum filling in the filling cavity of the vacuum chamber, in which air in the cavities in the microneedle female mold can be adequately displaced by the cast solution, ensuring that the cavities in the microneedle female mold can be filled up by the solution as a result of vacuum destruction and thus ensuring formation quality of the resulting microneedles.

In the above device and methods, the leveling mechanism can perform a leveling process on the solution cast on the surface of the microneedle female mold. In this way, the viscous solution can be sufficiently flattened on, so as to uniformly spread over, the surface of the microneedle female mold, thereby additionally ensuring formation quality of the resulting microneedles. In particular, the leveling process on the microneedle female mold may be effectuated by rotation of the leveling mechanism. An axis of rotation of the spindle may be oriented in parallel to a depthwise direction of the cavities in the microneedle female mold (also defined as an axial direction thereof), allowing the viscous solution to be flattened on the microneedle female mold under the action of rotation of the spindle.

After that, the viscous solution can move into the cavities in the microneedle female mold under the action of its own gravity. This allows the viscous solution to be flattened on the surface of the microneedle female mold in an effective and desirable manner without direct contact with the solution. Therefore, contamination of the solution can be avoided, additionally ensuring formation quality of the resulting microneedles.

### BRIEF DESCRIPTION OF THE DRAWINGS

Those of ordinary skill in the art would appreciate that the following drawings are presented merely to enable a better understanding of the present invention rather than to limit the scope thereof in any sense. In the drawings:

FIG. 1 shows a schematic front view of a device for producing microneedles according to a preferred embodiment of the present invention;

FIG. 2 is a schematic top view of the exterior of a vacuum chamber according to a preferred embodiment of the present invention;

FIG. 3 is a schematic diagram showing the internal structure of a vacuum chamber according to a preferred embodiment of the present invention;

FIG. 4 is a schematic top view of a device for producing microneedles according to a preferred embodiment of the present invention;

FIG. 5 is a front view of a leveling mechanism according to a preferred embodiment of the present invention;

FIG. 6 is a front view of a spindle assembly and a chuck assembly according to a preferred embodiment of the present invention;

FIG. 7 is a perspective view of a spindle assembly and a chuck assembly according to a preferred embodiment of the present invention;

FIG. 8 is a top view of a spindle assembly and a chuck assembly according to a preferred embodiment of the present invention;

FIG. 9 explains how unlocking of jaws is achieved as a result of extension of a pneumatic cylinder assembly according to a preferred embodiment of the present invention;

FIG. 10 explains how locking of jaws is achieved under the action of a tension spring in response to withdrawal of a pneumatic cylinder assembly according to a preferred embodiment of the present invention; and

FIG. 11 shows a rotational speed vs. time profile of a leveling mechanism during its operation according to a preferred embodiment of the present invention.

### DETAILED DESCRIPTION

The present invention will be described in greater detail below with reference to the accompanying schematic drawings, which present preferred embodiments of the invention. It would be appreciated that those skilled in the art can make changes to the invention disclosed herein while still obtaining the beneficial results thereof. Therefore, the following description shall be construed as being intended to be widely known by those skilled in the art rather than as limiting the invention.

For the sake of clarity, not all features of an actual implementation are described in this specification. In the following, description and details of well-known functions and structures are omitted to avoid unnecessarily obscuring the invention. It should be appreciated that in the development of any such actual implementation, numerous implementation-specific decisions must be made to achieve specific goals of the developers, such as compliance with system-related and business-related constrains, which may vary from one implementation to another. Moreover, it should be appreciated that such a development effort might be complex and time consuming, but would nevertheless be a routine undertaking for those of ordinary skill in the art.

The present invention will be described in greater detail below by way of examples with reference to the accompanying drawings. Advantages and features of the present invention will become more apparent from the following description. Note that the figures are provided in a very simplified form not necessarily drawn to exact scale for the only purpose of facilitating easy and clear description of the disclosed embodiments.

The present invention will be further described with reference to the accompanying drawings and to a few embodiments thereof.

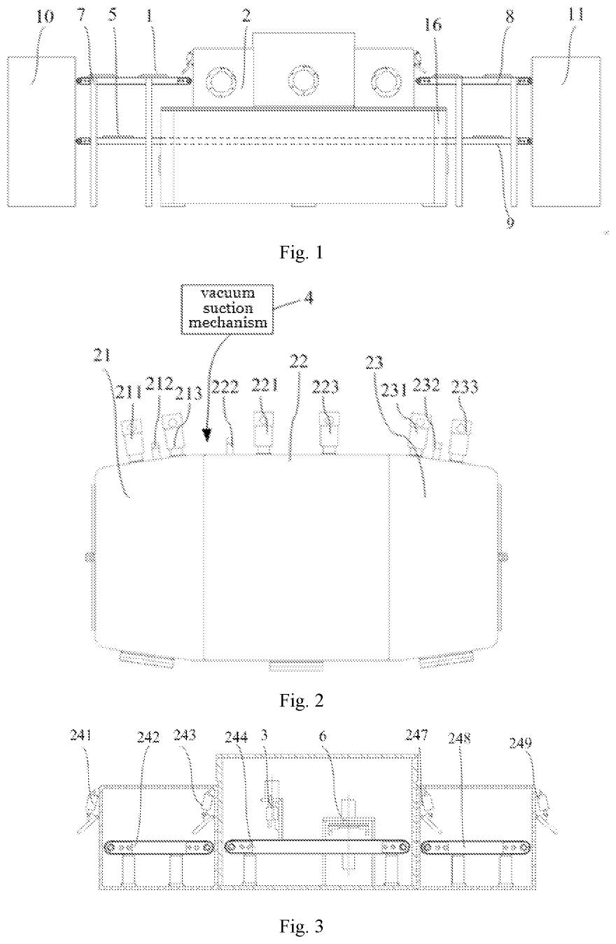

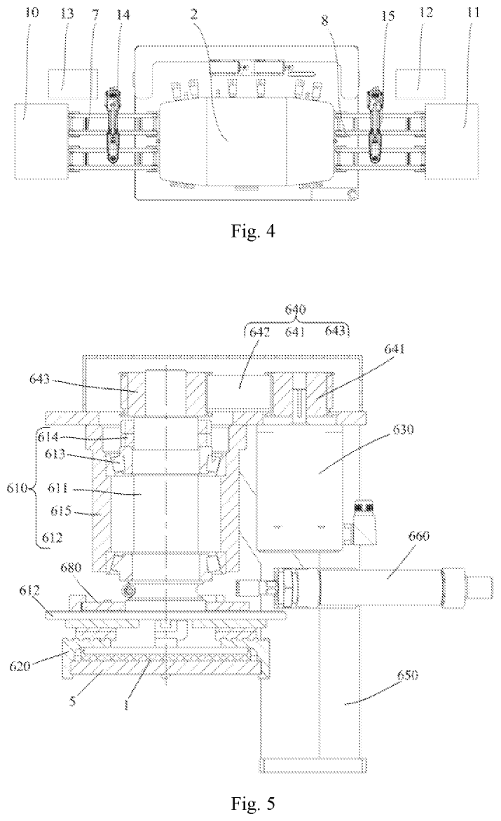

FIG. 1 shows a schematic front view of a device for producing microneedles according to a preferred embodiment of the present invention. FIG. 2 is a schematic top view of the exterior of a vacuum chamber according to a preferred embodiment of the present invention. FIG. 3 is a schematic diagram showing the internal structure of a vacuum chamber according to a preferred embodiment of the present invention. FIG. 4 is a schematic top view of a device for producing microneedles according to a preferred embodiment of the present invention.

As shown in FIGS. 1 to 4, embodiments of the present invention provide a device for producing microneedles, the microneedles are capable of piercing the stratum corneum of the human body to form channels, which facilitate drug delivery and transdermal drug absorption. The present application is not limited to any particular shape of the microneedles. As a non-limiting example, without limitation, the microneedles may have tip portions in the shape of protrusions, which may be either pointed or not. As a non-limiting example, the microneedles may be conical, pyramidal, or spindle-shaped. The microneedles are produced from a solution with a certain degree of viscosity. Examples of the solution may include, but are not limited to, polymer solutions, but the present application is not limited to any particular solution.

Specifically, the device includes a microneedle female mold 1, a vacuum chamber 2, a filling mechanism 3 and an evacuation mechanism 4. The microneedle female mold 1 is formed on its surface with grooves (not shown) matching with the microneedles to be produced. The filling mechanism 3 is at least partially disposed in the vacuum chamber and configured to release the solution for producing the microneedles. The vacuum chamber 2 is configured to provide a hermetic environment, where a vacuum can be created to facilitate the production of the microneedles. The evacuation mechanism 4 is connected to the vacuum chamber 2 and configured to evacuate the vacuum chamber 2.

As shown in FIG. 2, the vacuum chamber 2 defines three sequentially adjacent independent vacuum cavities, which are a feeding cavity 21, a filling cavity 22 and a discharging cavity 23. The feeding cavity 21 is configured to receive the microneedle female mold 1 therein under non-negative pressure, i.e., under atmospheric pressure. In the context herein, it would be appreciated that the atmospheric pressure is not necessarily equal to one standard atmosphere, but may deviate therefrom depending on the geographical location, altitude, temperature and/or other factors. Accordingly, the present application is not limited to any particular value of the atmospheric pressure. The evacuation mechanism 4 is configured to evacuate the feeding cavity 21 after the microneedle female mold 1 is received in the feeding cavity 21, so that both the microneedle female mold 1 and the feeding cavity 21 are under negative pressure. The filling cavity 22 is configured to receive therein, under negative pressure, the microneedle female mold 1 transported from the feeding cavity 21 also under negative pressure. The filling mechanism 3 is configured to cast the solution for producing the microneedles onto the surface of the microneedle female mold 1 in the filling cavity 22 under negative pressure, obtaining the microneedle female mold 1 with the solution cast thereon. Preferably, it is also configured to perform a leveling process on the microneedle female mold 1 with the solution cast thereon so that the solution is uniformly flattened on the surface of the microneedle female mold 1. The discharging cavity 23 is configured to receive therein, under negative pressure, the microneedle female mold 1 with the solution cast thereon from the filling cavity 22 also under negative pressure. After receiving the microneedle female mold 1 with the solution cast thereon, the discharging cavity 23 is also configured to destroy the vacuum so as to be under non-negative pressure, allowing the microneedle female mold 1 with the solution thereon to be taken out. At this time, the microneedle female mold 1 with the solution thereon is under atmospheric pressure. After the solution on the microneedle female mold 1 solidifies, the microneedles will be obtained (as integrated microneedles).

Embodiments of the present invention also provide a method for producing microneedles, which includes the steps as follows:

Step 11: Place the microneedle female mold 1 in the feeding cavity 21 under non-negative pressure and evacuate the feeding cavity 21 by the evacuation mechanism 4 so that both the feeding cavity 21 and the microneedle female mold 1 are under negative pressure.

Step 12: Transport the microneedle female mold 1 under negative pressure from the feeding cavity 21 under negative pressure into the filling cavity 22 under negative pressure and cast, under negative pressure, the solution onto the surface of the microneedle female mold 1 in the filling cavity 22 by the filling mechanism 3. Preferably, after the solution is cast, a leveling process is performed on the microneedle female mold 1 with the solution cast thereon by the leveling mechanism 6 so that the solution is uniformly flattened on the surface of the microneedle female mold 1.

Step 13: Transport the microneedle female mold 1 with the solution cast thereon from the filling cavity 22 under negative pressure into the discharging cavity 23 under negative pressure and maintain the filling cavity 22 under negative pressure. That is, after the microneedle female mold 1 with the solution cast thereon is transported out of the filling cavity 22, the filling cavity 22 is closed and, thereby the filling cavity 22 is maintained under negative pressure so as to be ready for the reception therein of the next microneedle female mold 1 to be casted thereon with the solution.

Step 14: After the microneedle female mold 1 with the solution cast thereon is transported into the discharging cavity 23 under negative pressure, destroy the vacuum in the discharging cavity 23 under negative pressure so that the discharging cavity 23 is under non-negative pressure, and take out the microneedle female mold 1 with the solution thereon. It would be appreciated that a vacuum has been drawn in the discharging cavity 23 before the microneedle female mold 1 with the solution cast thereon is received therein and that after the vacuum in the discharging cavity 23 is destroyed, the solution fills up the cavities of the microneedle female mold 1 by its own gravity.

Step 15: After the solution solidifies on the microneedle female mold, remove the mold, obtaining the microneedles.

Preferably, after step 14, the method further includes the steps detailed below.

After the vacuum in the discharging cavity 23 is destroyed, an exit of the discharging cavity 23 is opened, and the microneedle female mold 1 with the solution cast thereon is transported out of the discharging cavity 23, emptying the discharging cavity 23 that has undergone vacuum destruction. After the discharging cavity 23 that has undergone vacuum destruction is emptied, the discharging cavity 23 is closed, and vacuum suction begins therein until a vacuum of a predetermined degree is created within the discharging cavity 23, getting it ready for subsequent reception of another microneedle female mold 1 with a solution cast thereon.

In addition, in step 12, after the microneedle female mold 1 is transported out of the feeding cavity 21 under negative pressure, the feeding cavity 21 is closed, and destruction of the vacuum in the feeding cavity 21 begins until pressure in the feeding cavity 21 becomes equal to that of the surrounding environment, getting it reading for reception of the next microneedle female mold 1.

The three independent vacuum cavities can greatly reduce waiting times during filling operation and significantly increase vacuum filling efficiency in microneedle production. It would be appreciated that, if a single vacuum cavity were used, it would be necessary for it to experience repeated vacuum suction and destruction cycles. This would require not only a long filling time, which would lead to low filling efficiency, but also great energy consumption, which would lead to high product cost. According to the present invention, among the three independent vacuum cavities in the vacuum chamber, it is not necessary for the filling cavity 22 to go through repeated vacuum suction and destruction cycles, thus lowering energy consumption, reducing waiting times during filling and resulting in increased filling efficiency. Therefore, the three independent vacuum cavities can greatly speed up production and increase the throughput of the apparatus. In fact, the throughput of a single device can be increased to as high as 4 products per minute. In particular, since the microneedle female mold 1 has been subjected to vacuum suction for a long time before filling, air in the cavities of the microneedle female mold 1 can be greatly reduced, eventually ensuring formation quality of the resulting microneedles and increasing their yield. Moreover, filling the solution under a vacuum condition enables adequate evacuation of air from the cavities in the microneedle female mold, ensuring that these cavities are filled up by the solution after the vacuum in the discharging cavity 23 is destroyed and thereby ensuring formation quality of the produced microneedles. In particular, the vacuum condition, coupled with the leveling process performed by the leveling mechanism 6, allows the viscous solution to be effectively flattened on, and uniformly spread over, the surface of the microneedle female mold, thus additionally ensuring formation quality of the obtained microneedles.

In other embodiments, it is also possible to cast the solution under non-negative pressure and then perform a leveling process by the leveling mechanism 6, followed by successive vacuum suction and destruction. This can also enable desirable filling of the solution. Accordingly, embodiments of the present invention provided another method for producing microneedles, which includes:
in step 21, placing the microneedle female mold 1 in the filling cavity 22 under non-negative pressure and casting the solution onto the surface of the microneedle female mold 1 by the filling mechanism 3;
in step 22, performing a leveling process on the microneedle female mold 1 by the leveling mechanism 6 so that the solution is uniformly flattened on the surface of the microneedle female mold 1;
in step 23, drawing a vacuum in the filling cavity 22 by the evacuation mechanism 4 so that both the filling cavity 22 and the microneedle female mold 1 are under negative pressure;
in step 24, after the microneedle female mold 1 that has undergone the leveling process is transported into the discharging cavity 23 under negative pressure, destroying the vacuum in the discharging cavity 23 under negative pressure so that the discharging cavity 23 is under non-negative pressure and taking out the microneedle female mold 1 with the solution thereon; and
in step 25, after the solution solidifies on the microneedle female mold 1, removing the mold.

It would be appreciated that vacuum pressure in the various vacuum cavities is predetermined according to the microneedle product being produced. Vacuum pressure in each vacuum cavity is required to satisfy a predefined condition. Insufficient vacuum pressure will affect formation quality of the produced microneedles, while excessive vacuum pressure will increase energy consumption, extend the times required for vacuum suction and destruction and reduce the throughput of the apparatus. In preferred embodiments, the feeding cavity 21, the filling cavity 22 and the discharging cavity 23 operate under equal pressure, which is preferred to range from −95 kPa to −80 kPa. This operating pressure can not only ensure formation quality of the resulting microneedles, but can also shorten the times required for vacuum suction and destruction and improve the apparatus' throughput. Further, the operating pressure of the feeding cavity 21, the filling cavity 22 and the discharging cavity 23 may be controlled at a resolution of 1 kPa. More preferably, vacuum suction in the feeding cavity 21 and the discharging cavity 23 takes 10-16 seconds, and vacuum destruction in the feeding cavity 21 and the discharging cavity 23 takes 3-5 seconds. As the filling cavity 22 is always kept under negative pressure, its vacuum suction and destruction is not associated with any particular time requirements.

Preferably, the device for producing the microneedle may further include a tray 5, the tray 5 is used for carrying the microneedle female mold 1 successively through the feeding cavity 21, the filling cavity 22 and the discharging cavity 23. More preferably, the same tray 5 can be used to transport microneedle female molds 1 of different sizes.

Further, use of the vacuum chamber 5 preferably involves: first of all, transporting the microneedle female mold 1 in the tray 5 into the feeding cavity 21 under atmospheric pressure; then closing the feeding cavity 21, thereby isolating the feeding cavity 21 from the other two vacuum cavities and the surrounding environment; subsequently, starting vacuum suction in the feeding cavity 21, stopping vacuum suction upon vacuum pressure therein reaching a predetermined value, and maintaining the negative pressure; after that, opening an exit of the feeding cavity 21, transporting the tray 5 together with the microneedle female mold 1 into the filling cavity 22, and closing the filling cavity 22, thereby isolating the filling cavity 22 from the other two vacuum cavities and the surrounding environment, during which, the filling cavity 22 has undergone vacuum suction before placing the microneedle female mold 1 therein; after the solution is cast onto the microneedle female mold 1, opening an exit of the filling cavity 22, transporting the tray 5 together with the microneedle female mold 1 with the solution casted therein into the discharging cavity 23, and closing the discharging cavity 23, thereby isolating the discharging cavity 23 from the other two vacuum cavities and the surrounding environment; and then destroying the vacuum in the discharging cavity 23 so that the pressure in the discharging cavity 23 becomes equal to that of the surrounding environment.

In addition, in step 13, after the solution is cast onto the microneedle female mold 1, a leveling process may be performed in the filling cavity 22, and the exit of the filling cavity 22 may be opened after the leveling process is completed. That is, the device for producing the microneedles may further include a leveling mechanism 6, and the filling cavity 22 preferably defines a casting location and a leveling location. With the filling cavity 22 being under negative pressure, the microneedle female mold 1 may be transported in the tray 5 to the casting location, and the filling mechanism 3 may then cast the solution onto the surface of the microneedle female mold 1 under negative pressure. After the solution is casted onto the microneedle female mold 1, the microneedle female mold 1 may be further transported in the tray 5 to the leveling location, where the leveling mechanism 6 may perform a leveling process on the microneedle female mold 1 with the solution cast thereon to allow the solution to uniformly spread over the surface of the microneedle female mold 1.

In embodiments of the present invention, the leveling mechanism 6 mainly serves to cause movement of the tray 5 as required to perform a leveling process on the microneedle female mold 1. The movement of the tray 5 may be any movement, for example, including at least one of horizontal rotation, horizontal translation, shaking and vertical swinging. In this arrangement, the leveling mechanism 6 will not contact the solution for producing the microneedles, reducing the risk of the solution being contaminated. Moreover, good leveling quality can be obtained with a relatively simple structure and easy operation, ensuring that a stable and consistent amount of the solution is filled in each cavity and thereby ensuring formation quality of the resulting microneedles.

In some embodiments, the leveling mechanism 6 includes a picking structure and a driving mechanism. The driving mechanism is configured to drive the picking structure to horizontally rotate about its own axis, and the picking structure is configured to pick up the tray 5. The driving mechanism then causes the picking structure together with the tray 5 and the microneedle female mold 1 to horizontally pivot, thereby centrifugally causing the solution to uniformly spread over the surface of the microneedle female mold 1 and fill up the cavities therein. Both the leveling mechanism 6 and the filling mechanism 3 may be disposed within the filling cavity 22. Alternatively, the leveling mechanism 6 may be disposed alone within a separate independent vacuum cavity arranged between the filling cavity 22 and the discharging cavity 23.

In some other embodiments, the leveling mechanism 6 includes a picking structure and a driving mechanism. The driving mechanism is configured to drive the picking structure to vertically swing, and the picking structure is configured to pick up the tray 5. The driving mechanism then causes the picking structure together with the tray 5 and the microneedle female mold 1 to vertically swing, thereby causing the solution to uniformly spread over the surface of the microneedle female mold 1 and fill up the cavities therein. In other embodiments, the picking structure may be omitted, and the driving mechanism may be configured instead as a moving table for carrying the tray 5 and driving the tray 5 to horizontally rotate, or vertically swing, or shake (including vibrating), or horizontally translate.

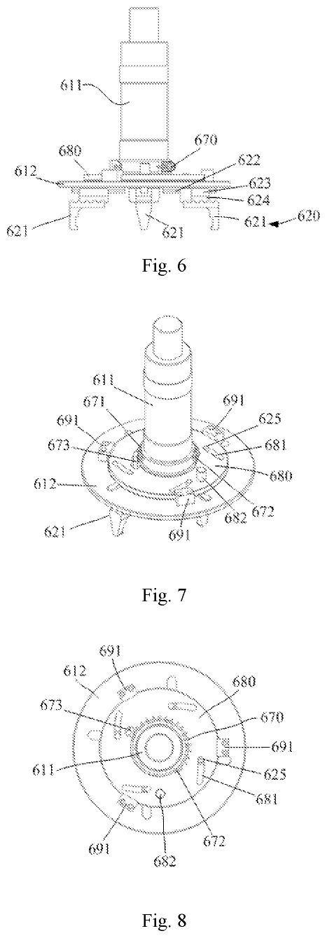

FIGS. 5 to 10 show a leveling mechanism 6 according to a preferred embodiment. The leveling mechanism 6 includes a picking structure and a driving mechanism. The picking structure includes a spindle assembly 610 and a chuck assembly 620. The driving mechanism includes servo motor 630 and a transmission assembly 640. The spindle assembly 610 includes a spindle 611 and a base 612. The bottom of the spindle 611 is coupled to the base 612 fixedly or detachably. The chuck assembly 620 includes at least three jaws 621, the at least three jaws 621 are disposed on the base 612 evenly about an axis of the spindle 611. The at least three jaws 621 can work together to clamp and retain the tray 5. Preferably, there are four jaws 621, which can provide consistent clamping. Each jaw 621 can move relative to the base 612 to clamp or release the tray 5. The servo motor 630 is connected to the spindle 611 via the transmission assembly 640 so that the transmission assembly 640 can drive the spindle 611 to horizontally rotate about its own axis. The transmission assembly 640 is preferred to be a belt/pulley assembly. Specifically, the transmission assembly 640 includes a driving pulley 641, a belt 642 and a driven pulley 643. The driving pulley 641 is coupled to a shaft of the (not labeled) of the servo motor 630. The belt 642 is wound on the driving pulley 641 and the driven pulley 643. The driven pulley 643 is disposed over a top portion of the spindle 611. Preferably, the servo motor 630 is disposed in parallel to the spindle 611. The servo motor 630 drives the spindle 611 and the base 612 to rotate through the belt/pulley assembly. As a result, the tray 5 rotates together with the microneedle female mold 1, allowing the solution to be uniformly coated on the surface of the microneedle female mold 1. The spindle 611 may be fixed to a bearing housing 615 through bearings 613 and nuts 614. Both the bearing housing 615 and the servo motor 630 are fixed to a frame 650. The base 612 may be of various shapes, and is preferred to be circular. It would be appreciated that an axis of rotation of the spindle 611 runs parallel to a depthwise direction of the cavities in the microneedle female mold. That is, the axis of rotation of the spindle 611 is parallel to axes of the cavities. Thus, rotation of the spindle 611 can flatten the viscous solution (for producing the microneedles) on the microneedle female mold, and the viscous solution can move into the cavities in the microneedle female mold by its own gravity. This allows the viscous solution to be flattened on the surface of the microneedle female mold in an effective and desirable manner without direct contact with the solution. Therefore, contamination of the solution can be avoided, additionally ensuring formation quality of the resulting microneedles.

More preferably, the driving mechanism further includes a pneumatic cylinder assembly 660, a resilient member 670 and a turntable 680. The turntable 680 is disposed over the spindle 611 so as to be rotatable relative to the spindle 611. One end of the resilient member 670 is coupled to the spindle 611, and the other end is coupled to the turntable 680. When the turntable 680 is driven by the pneumatic cylinder assembly 660 to rotate in a first direction, the resilient member 670 will store resilient energy and switch the jaws 621 of the chuck assembly 620 to respective unlocking positions, thereby releasing the tray 5 that they retain, or allowing the tray 5 to be placed between them and subsequently clamped and retained thereby. When the pneumatic cylinder assembly 660 stops acting on the turntable 680, the resilient member 670 will release the resilient energy that it stores, causing the turntable 680 to rotate in a second direction. As a result, the jaws 621 of the chuck assembly 620 are moved to respective locking positions, thereby clamping the tray 5, or returning to their rest positions after the tray 5 is released therefrom. Locking and unlocking the jaws 621 in this way under the action of the pneumatic cylinder and the energy storage element dispenses with the use of complex supporting devices such as pneumatic and electric circuits, resulting in structural simplicity and high reliability. Moreover, the spindle 611 may be rotated at a desired speed under the control of the servo motor 630, providing a desired centrifugal effect. The resilient member 670 is generally selected as a tension spring 671, one end of the resilient member 670 is fixed to the spindle 611 and the other end thereof is fixed to the turntable 680. In one embodiment, one end of the tension spring 671 is fixed to a first pin 672, the first pin 672 is fixed to the spindle 611. The other end of the tension spring 671 is fixed to a second pin 673, the second pin 673 is fixed to the turntable 680. The first direction is opposite to the second direction.

In order to avoid unwanted wobbling of the turntable 680, the leveling mechanism 6 may further include an auxiliary member 690, the auxiliary member 690 is used for restricting the position of the turntable 680 axially. Optionally, the auxiliary member 690 may include press blocks 691, the press blocks 691 are fixed to the base 612 and configured to axially press against the turntable 680. At least three press blocks 691 may be provided on the base 612 and circumferentially evenly spaced from one another.

The chuck assembly 620 may include guide rails 622, sliders 623, fixation bases 624 and stop pins 625. The guide rails 622 extend radially with respect to the base 612, and the sliders 623 are slidably disposed on the guide rails 622. The fixation bases 624 are attached to the sliders 623, and each jaw 621 is secured to a respective one of the fixation bases 624. The stop pins 625 are fixed to the fixation bases 624, and the turntable 680 defines curved stop slots 681. The stop pins 625 are movably inserted in the curved stop slots 681. Opposite ends of each curved stop slot 681 are spaced from the center of the turntable 680 at different distances. Before a clamping configuration, the chuck assembly 620 is in an initial configuration, the stop pins 625 abut against proximal ends of the curved stop slots 681, which are closer to the center of the turntable than distal ends of the curved stop slots 681. As the turntable 680 is rotated in the first direction, the stop pins 625 move from the proximal ends of the stop slots 681 towards the distal ends thereof, urging the jaws 621 outwards. The stop pins 625 stop upon coming into abutment against the distal ends of the stop slots 681. As the turntable 680 is rotated in the second direction, the stop pins 625 move from the distal ends of the stop slots 681 towards the proximal ends thereof, urging the jaws 621 inwards. The stop pins 625 stop upon coming into abutment against the proximal ends of the stop slots 681.

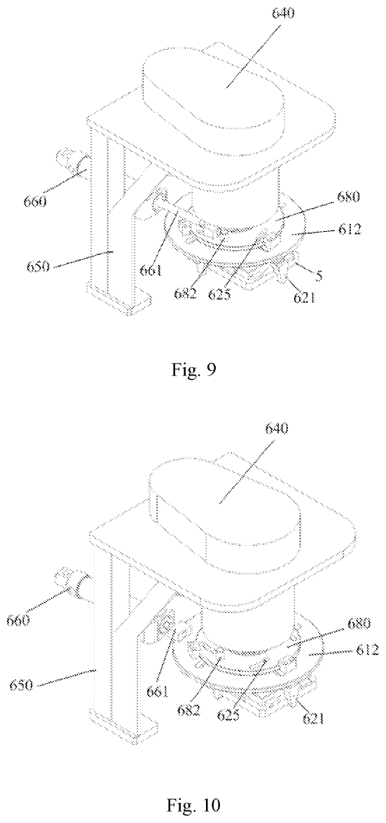

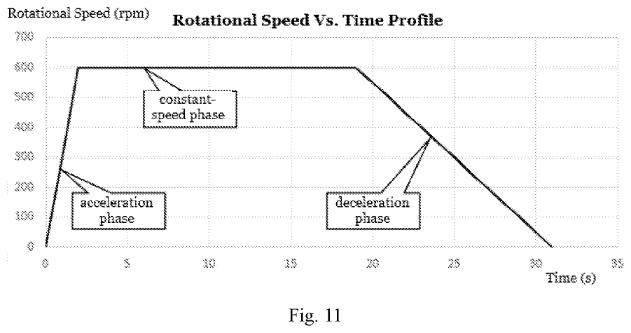

With combined reference to FIGS. 7 to 8, the pneumatic cylinder assembly 660 may include a push rod 661, and a fixed stud 682 may be provided on the turntable 680. As shown in FIG. 9, in order to unlock the jaws 621, the pneumatic cylinder drives the push rod 661 to extend outwards until it comes into abutment against the fixed stud 682. After that, it further pushes the fixed stud 682, thereby rotating the turntable 680 in the first direction. As a result, the stop pins 625 urge the fixation bases 624 to radially slide outwards on the guide rails 622, unlocking the tray 5 from the jaws 621. As shown in FIG. 10, once the push rod 661 is withdrawn, the turntable 680 is biased by the tension spring 671 to rotate in the second direction. As a result, the stop pins 625 urge the fixation bases 624 to radially slide inwards on the guide rails 622, causing the jaws 621 to lock the tray 5.

In order to provide an additional leveling effect, according to embodiments of the present invention, operation of the leveling mechanism 6 may be divided into several stages: acceleration, constant-speed and deceleration. Accordingly, in the leveling process performed by the leveling mechanism 6 on the microneedle female mold 1, it may rotate successively at a rising speed, a constant speed and a decreasing speed, in order to allow the solution to uniformly spread over the surface of the microneedle female mold. FIG. 11 shows a rotational speed vs. time profile of the leveling mechanism 6, in which abscissa represents time (in seconds) and the ordinate represents rotational speed (in rpm). In practical use, the spindle 611 may first rotate at an increasing speed. When the rotational speed of the spindle 611 reaches a predetermined value, it may be maintained for a period of time. After that, the spindle 611 may rotate at a decreasing speed until the leveling process ends. Since the rotational speed of the spindle 611 and the duration of the leveling process may vary depending on the viscosity of the solution for producing the microneedles, the speeds and durations of the various stages may be determined according to the material and viscosity of the microneedles, and the present application is not limited to any particular values thereof. Optionally, the rotational speed in the constant-speed stage may be 300-1000 rpm, and the acceleration stage may end within 2 seconds. Additionally, the constant-speed stage may last for 2-18 seconds, and the deceleration stage may last for 18-32 seconds.

The device for producing the microneedles may further include conveyors for transporting the tray 5 in an automated manner and enabling reuse of the tray 5. The device for producing the microneedles may define a loading zone and an unloading zone, the loading zone is arranged on the side of the feeding cavity 21 and the unloading zone is arranged on the side of the discharging cavity 23. The conveyor may continuously transport the tray 5 from the unloading zone to the loading zone, enabling reuse of the tray 5.

As shown in FIG. 1, the conveyors may include a loading conveyor 7, an unloading conveyor 8 and a transfer conveyor 9. The loading conveyor 7 is arranged in the loading zone, i.e., at an entrance of the vacuum chamber 5. The unloading conveyor 8 is arranged in the unloading zone, i.e., at an exit of the vacuum chamber 5. That is, the vacuum chamber 2 is located between the loading conveyor 7 and the unloading conveyor 8.

The transfer conveyor 9 may be directly connected to the loading conveyor 7 and the unloading conveyor 8, forming a complete, continuous, endless conveyor. Alternatively, the transfer conveyor 9 may not be connected to the loading conveyor 7 and the unloading conveyor 8, forming a discontinuous, endless conveyor. In this case, the tray 5 may be transported by an automated transport device from the unloading conveyor 8 to the transfer conveyor 9, and then the tray 5 may be transported by an automated transport device from the transfer conveyor 9 to the loading conveyor 7. In this way, a microneedle production loop with a high degree of automation can be formed, which can greatly speed up production and increase the apparatus' throughput.

In addition, the loading conveyor 7 may transport the microneedle female mold 1 in the tray 5 to the feeding cavity 21 under atmospheric pressure, and the unloading conveyor 8 may receive the tray 5 from the discharging cavity 23. The transfer conveyor 9 may receive the empty tray 5 from the unloading conveyor 8 and the transfer conveyor 9 may transport the empty tray 5 to the loading zone. The present invention is not limited to any particular position of the transfer conveyor 9 relative to the loading conveyor 7, or to the unloading conveyor 8. In the embodiment shown in FIG. 1, the transfer conveyor 9 is disposed under the loading conveyor 7 and the unloading conveyor 8, for space-saving purposes. Accordingly, the device may further include a support frame 16 configured for arrangement of the vacuum chamber 2 and the conveyor thereon.

Preferably, the device for producing the microneedles further includes an automated transport device for transporting the empty tray 5 from the unloading conveyor 8 to the transfer conveyor 9, and the automated transport device is used for transporting the empty tray 5 from the transfer conveyor 9 to the loading conveyor 7. The automated transport device is preferred to be a hoist device, the hoist device includes a loading hoist 10 and an unloading hoist 11. The loading hoist 10 is disposed in the loading zone, and the unloading hoist 11 is arranged in the unloading zone. In this case, the transfer conveyor 9 may be arranged at a different height from the loading conveyor 7 and the unloading conveyor 8.

For example, in the embodiment shown in FIG. 1, the transfer conveyor 9 is arranged under, for example, directly under the loading conveyor 7 and the unloading conveyor 8. The loading hoist 10 may provide a transition between the transfer conveyor 9 and the loading conveyor 7, and the unloading hoist 11 may provide a transition between the transfer conveyor 9 and the unloading conveyor 8. The loading hoist 10 may be configured to transport the empty tray 5 from the transfer conveyor 9 to the loading conveyor 7 by automated raising and lowering, and the unloading hoist 11 may be configured to transport the empty tray 5 from the unloading conveyor 8 to the transfer conveyor 9 by automated raising and lowering. Of course, the automated transport device is not limited to being implemented as the hoists, and it may also be implemented as, for example, robotic arms, automated guided vehicles, automation tracks, or the like.

Further, a step in a microneedle production process using the above device for producing the microneedles may include:
first of all, in the loading zone, placing the microneedle female mold 1 into the empty tray 5 on the loading conveyor 7 manually, or by mechanical automation; after the tray 5 is loaded in the microneedle female mold 1, transporting it by the loading conveyor 7 into the feeding cavity 21 of the vacuum chamber 2; after the solution is cast onto the microneedle female mold 1 and a leveling process is performed thereon, transporting it in the tray 5 into the discharging cavity 23 and destroying a vacuum therein; after the vacuum is destroyed, transporting the microneedle female mold 1 with the solution cast thereon in the tray 5 onto the unloading conveyor 8; and in the unloading zone, taking out the microneedle female mold 1 with the solution cast thereon out of the tray 5 manually, or by mechanical automation. After the microneedle female mold 1 with the solution cast thereon is taken out from the tray 5, it is usually placed into a transfer tray 12 and then transported therein to the next process step (e.g., drying or baking). After the microneedle female mold 1 with the solution cast thereon is taken out from the tray 5, the empty tray 5 may be transported by the unloading hoist 11 onto the transfer conveyor 9, the transfer conveyor 9 may then transport the empty tray 5 further into the loading zone. After arriving at the loading zone, the loading hoist 10 may transport the empty tray 5 onto the loading conveyor 7, getting it ready for reception of the next mold.

With continued reference to FIG. 2, the feeding cavity 21 may be provided with a first vacuum suction valve 211. The first vacuum suction valve 211 is in communication with the feeding cavity 21 and the first vacuum suction valve 211 is connected to the evacuation mechanism 4. The evacuation mechanism 4 may evacuate the feeding cavity 21 through the first vacuum suction valve 211, thereby maintaining the feeding cavity 21 under negative pressure. The device for producing the microneedles may further include a sensor assembly including a first sensor 212 provided in the feeding cavity 21. The first sensor 212 is configured for real-time detection of a degree of vacuum in the feeding cavity 21.

Preferably, the first sensor 212 is communicatively connected to a controller and feeds the detected degree of vacuum back to the controller, allowing the controller to control pressure in the feeding cavity 21 based on the feedback. The feeding cavity 21 may be provided with a first vacuum breaker valve 213, which can communicate with the surrounding environment and thereby destroy the vacuum in the feeding cavity 21. Preferably, the first vacuum breaker valve 213 is communicatively connected to the controller so as to be able to be activated and deactivated under the control of the controller. Preferably, the first vacuum suction valve 211 is communicatively connected to the controller so as to be able to be activated and deactivated under the control of the controller.

FIG. 2 also shows the filling cavity 22 according to an exemplary embodiment. The filling cavity 22 may be provided with a second vacuum suction valve 221 in communication with the filling cavity 22 and connected to the evacuation mechanism 4. The evacuation mechanism 4 may evacuate the filling cavity 22 through the second vacuum suction valve 221, thereby maintaining the filling cavity 22 under negative pressure. Preferably, the sensor assembly further includes a second sensor 222 provided in the filling cavity 22. The second sensor 222 is configured for real-time detection of a degree of vacuum in the filling cavity 22. Preferably, the second sensor 222 is communicatively connected to the controller and feeds the detected degree of vacuum back to the controller, allowing the controller to control pressure in the filling cavity 22 based on the feedback. The filling cavity 22 may be provided with a second vacuum breaker valve 223, which can communicate with the surrounding environment and thereby destroy the vacuum in the filling cavity 22. Preferably, the second vacuum breaker valve 223 is communicatively connected to the controller so as to be able to be activated and deactivated under the control of the controller. Preferably, the second vacuum suction valve 221 is communicatively connected to the controller so as to be able to be activated and deactivated under the control of the controller.

FIG. 2 also shows the discharging cavity 23 according to an exemplary embodiment. The discharging cavity 23 may be provided with a third vacuum suction valve 231 in communication with the discharging cavity 23 and connected to the evacuation mechanism 4. The evacuation mechanism 4 may evacuate the discharging cavity 23 through the third vacuum suction valve 231, thereby maintaining the discharging cavity 232 under negative pressure. Preferably, the sensor assembly further includes a third sensor 232 provided in the discharging cavity 23. The third sensor 232 is configured for real-time detection of a degree of vacuum in the discharging cavity 23. Preferably, the third sensor 232 is communicatively connected to the controller and feeds the detected degree of vacuum back to the controller, allowing the controller to control pressure in the discharging cavity 23 based on the feedback. The discharging cavity 23 may be provided with a third vacuum breaker valve 233, which can communicate with the surrounding environment and thereby destroy the vacuum in the discharging cavity 23. Preferably, the third vacuum breaker valve 233 is communicatively connected to the controller so as to be able to be activated and deactivated under the control of the controller. Preferably, the third vacuum suction valve 231 is communicatively connected to the controller so as to be able to be activated and deactivated under the control of the controller.

FIG. 3 shows the internal structure of the vacuum chamber 2 according to an exemplary embodiment. A first hatch 241 may be provided at an entrance of the feeding cavity 21, and a common second hatch 243 may be preferably provided at both the exit of the feeding cavity 21 and an entrance of the filling cavity 22. A common third hatch 247 may be preferably provided at both the exit of the filling cavity 22 and an entrance of the discharging cavity 23, and a fourth hatch 249 may be provided at the exit of the discharging cavity 23. The first hatch 241 may be configured to open and close the entrance of the feeding cavity 21. The second hatch 243 may be configured to bring the feeding cavity 21 and the filling cavity 22 into communication with each other and separate them from each other. The third hatch 247 may be configured to bring the filling cavity 22 and the discharging cavity 23 into communication with each other and separate them from each other. The fourth hatch 249 may be configured to open and close the exit of the discharging cavity 23.

Use of the aforementioned hatches may include the steps of: opening the first hatch 241 when the feeding cavity 21 is under atmospheric pressure, transporting the tray 5 carrying the microneedle female mold 1 onto a first conveyor 242 provided in the feeding cavity 21, closing the first hatch 241, and starting drawing a vacuum in the feeding cavity 21; after the vacuum suction is completed, opening the second hatch 243 and transporting the tray 5 through the first conveyor 242 and a second conveyor 244 into the filling cavity 22, where the second conveyor 244 is provided in the filling cavity 22; upon the tray 5 reaching an operating location of the filling mechanism 3, closing the second hatch 243, destroying the vacuum in the feeding cavity 21 after the second hatch 243 is closed, and opening the first hatch 241 after the vacuum in the feeding cavity 21 is destroyed, getting it ready for reception of the next tray 5; with the tray 5 being situated at the operating location of the filling mechanism 3, activating the filling mechanism 3 which then casts a predetermined amount of the solution onto the surface of the microneedle female mold 1, and after the filling is completed, transporting the tray 5 through the second conveyor 244 to an operating location of the leveling mechanism 6; upon the tray 5 arriving at the leveling location, performing a leveling process by the leveling mechanism 6 on the microneedle female mold 1 so that the solution rapidly and uniformly spreads over the entire surface of the microneedle female mold 1; after the leveling process is completed, opening the third hatch 247 and transporting the tray 5 through the second conveyor 244 and a third conveyor 248 into the discharging cavity 23, wherein the third conveyor 248 is provided in the discharging cavity 22; after the tray 5 reaches a desired location, closing the third hatch 247 and then destroying a vacuum in the discharging cavity 23 until pressure in the discharging cavity 23 becomes equal to that of the outside world; and opening the fourth hatch 249, transporting the tray 5 out of the discharging cavity 23 through the third conveyor 248, closing the fourth hatch 249 and starting drawing a vacuum in the discharging cavity 23 until a predetermined degree of vacuum is attained therein.

Preferably, the device for producing the microneedles further includes a controller, which is preferably communicatively connected to the vacuum suction valves, the vacuum breaker valves, the evacuation mechanism 4 and the sensor assembly so as to be able to control automated operation of these components. Preferably, the evacuation mechanism 4 includes a first vacuum suction pump, a second vacuum suction pump and a third vacuum suction pump. The first vacuum suction pump is configured to evacuate the feeding cavity 21 through the first vacuum suction valve 211. The second vacuum suction pump is configured to evacuate the filling cavity 22 through the second vacuum suction valve 221. The third vacuum suction pump is configured to evacuate the discharging cavity 23 through the third vacuum suction valve 231. The controller is configured to: open the first vacuum breaker valve 213 to destroy a vacuum in the feeding cavity 21; open the second vacuum breaker valve 223 to destroy a vacuum in the filling cavity; and open the third vacuum breaker valve 233 to destroy a vacuum in the discharging cavity 23. Preferably, the controller is configured to control, based on detected information from the first sensor 212, the second sensor 222 and the third sensor 232, degrees of vacuum in the respective cavities. That is, it is configured to control vacuum suction of the vacuum pumps based on the detected information from the respective sensors so that desired degrees of vacuum are achieved. Further, after the microneedle female mold 1 in a fourth state is taken out of the discharging cavity 23 that has experienced vacuum destruction, the controller may control the third vacuum suction pump to evacuate the discharging cavity 23. Furthermore, after the microneedle female mold 1 under negative pressure is transported into the filling cavity 22 under negative pressure, the controller may open the first vacuum breaker valve 213 to destroy the vacuum in the feeding cavity 21.

As shown in FIG. 4, in order to achieve a further higher degree of automation, the device for producing the microneedles is preferred to further include automated feeding mechanism 13 and an automated loading mechanism 14, both disposed in the loading zone. With the automated feeding mechanism 13 and the automated loading mechanism 14, automated loading of the microneedle female mold 1 can be accomplished without involving manual intervention.

The automated feeding mechanism 13 is configured to transport the microneedle female mold 1 under atmospheric pressure to a loading location in an automated manner. The automated loading mechanism 14 is configured to pick up the microneedle female mold 1 under atmospheric pressure from the loading location and place it into the tray 5 on the loading conveyor 7 in an automated manner. The present application is not limited to any particular implementation of the automated feeding mechanism 13, and for example, it may be implemented as an automated lifting platform designed with multiple layers, each of which may be placed thereon with one or more microneedle female molds 1 to be filled. Preferably, the automated loading mechanism 14 is implemented as a loading robotic arm, which features flexibility and convenience of operation and is space-saving.

With continued reference to FIG. 5, the device for producing the microneedles is preferred to further include an automated unloading mechanism 15 and a transfer tray 12, both disposed in the unloading zone. The automated unloading mechanism 15 is configured to pick up the microneedle female mold 1 in the fourth state from the tray 5 on the unloading conveyor 8 and place it into the transfer tray 12. Thus, the automated unloading mechanism 15 is able to accomplish unloading of the microneedle female mold 1 without involving manual intervention, additionally enhancing automation of microneedle production. In the unloading zone, the automated unloading mechanism 15 may pick up the filled microneedle female mold 1 from the tray 5 and place it in a transfer zone in an automated manner. For example, the transfer tray 12 may be disposed in the transfer zone, and the automated unloading mechanism 15 may place the filled microneedle female mold 1 into the transfer tray 12. Subsequently, microneedle female molds 1 may be removed manually from such transfer trays 12 on a regular basis. In this way, a single operator is enabled to manage multiple apparatuses, significantly reducing labor investment and labor cost. The automated unloading mechanism 12 is usually implemented as an unloading robotic arm.

The present application is not limited to any particular implementation of the filling mechanism 3. The filling mechanism 3 may include a dispensing nozzle for releasing the solution for producing the microneedles. Preferably, multiple such dispensing nozzles are included and arranged into a row. Each of the dispensing nozzle is configured to release the solution. Simultaneous release of the solution from the multiple dispensing nozzles can achieve better solution casting with higher efficiency.

The present invention is not limited to any particular type of controller, and the controller may be any suitable hardware device that performs logical operations, such as a single-chip microcomputer, a microprocessor, a programmable logic controller (PLC) or a field-programmable gate array (FPGA), or any suitable software program, functional module, function, object library or dynamic-link library that performs the above functions on the basis of hardware, or a combination of both. On the basis of the teachings disclosed herein, those skilled in the art would know how to achieve the communication between the controller and the other components. Although the use of a controller has been described as being preferred in the above embodiments, those skilled in the art can use alternative technical means such as manual control and mechanical control while still achieving the same beneficial results.

The description presented above is merely that of a few preferred embodiments of the present invention and is not intended to limit the scope thereof in any sense. Any and all changes and modifications made by those of ordinary skill in the art based on the above teachings fall within the scope of the invention.

1. A device for producing microneedles, comprising a microneedle female mold, a vacuum chamber, a filling mechanism and an evacuation mechanism, a surface of the microneedle female mold provided with grooves matching with the microneedles, the filling mechanism at least partially disposed in the vacuum chamber and configured to release a solution for producing the microneedles, the evacuation mechanism connected to the vacuum chamber and configured to evacuate the vacuum chamber, the vacuum chamber comprising a feeding cavity, a filling cavity and a discharging cavity, which are independent of one another,
the feeding cavity configured to receive the microneedle female mold in a non-negative pressure state,
the evacuation mechanism configured to evacuate the feeding cavity that has received the microneedle female mold therein,
the filling cavity configured to receive, in a negative pressure state, the microneedle female mold transported from the feeding cavity in the negative pressure state,
the discharging cavity configured to receive, in the negative pressure state, the microneedle female mold transported from the filling cavity in the negative pressure state and to undergo vacuum destruction after the microneedle female mold is received therein.

2. The device for producing microneedles according to claim 1, further comprising a controller and vacuum breaker valves communicatively connected to the controller, the vacuum breaker valves including a first vacuum breaker valve and a third vacuum breaker valve, the first vacuum breaker valve disposed in the feeding cavity, the third vacuum breaker valve disposed in the discharging cavity,
the controller configured to control an opening of the first vacuum breaker valve to break vacuum in the feeding cavity and to control an opening of the third vacuum breaker valve to break vacuum in the discharging cavity.

3. The device for producing microneedles according to claim 2, wherein the controller is further communicatively connected to the evacuation mechanism, and wherein the controller is configured to control the evacuation mechanism to evacuate the discharging cavity, the filling cavity and the feeding cavity.

4. The device for producing microneedles according to claim 3, further comprising a sensor assembly communicatively connected to the controller, the sensor assembly comprising a first sensor, a second sensor and a third sensor,
wherein the feeding cavity is provided with the first sensor; the filling cavity is provided with the second sensor; the discharging cavity is provided with the third sensor; and the controller is configured to control, based on pressure information detected by the first sensor, the second sensor and the third sensor, degrees of vacuum in the respective cavities.

5. The device for producing microneedles according to claim 1, further comprising a leveling mechanism, wherein the filling cavity defines therein a casting location and a leveling location;
when the filling cavity is in the negative pressure state and when the microneedle female mold is located at the casting location in the filling cavity, the filling mechanism is configured to cast the solution onto the surface of the microneedle female mold;
after the solution is cast onto the microneedle female mold in the filling cavity, the microneedle female mold is configured to be transported to the leveling location, and the leveling mechanism is configured to perform a leveling process on the microneedle female mold with the solution cast thereon, so that the solution uniformly spreads over the surface of the microneedle female mold.

6. The device for producing microneedles according to claim 5, wherein the leveling mechanism comprises a picking structure and a driving mechanism, the picking structure comprising a spindle assembly and a chuck assembly, the driving mechanism comprising a servo motor and a transmission assembly, the spindle assembly comprising a spindle and a base, a bottom of the spindle fixedly connected to the base, the chuck assembly comprising at least three jaws, which are arranged on the base evenly about an axis of the spindle and configured to clamp and retain a tray, the tray used for carrying the microneedle female mold therein, the servo motor configured to drive the spindle to rotate through the transmission assembly, the axis of the spindle parallel to axes of the cavities.

7. The device for producing microneedles according to claim 6, wherein the driving mechanism further comprises a pneumatic cylinder assembly, a resilient member and a turntable, the turntable disposed over the spindle so as to be rotatable relative to the spindle, one end of the resilient member connected to the spindle and the other end of the resilient member connected to the turntable,
wherein as a result of the turntable being driven by the pneumatic cylinder assembly to rotate in a first direction, the resilient member stores resilient energy, causing all the jaws to move to their unlocking positions; and
when the pneumatic cylinder assembly stops acting on the turntable, the resilient member releases the stored resilient energy, driving the turntable to rotate in a second direction and causing all the jaws to move to their locking positions.

8. The device for producing microneedles according to claim 7, wherein the leveling mechanism further comprises press blocks, which are fixed to the base and configured to axially press against the turntable, there are at least three press blocks; and/or
wherein the chuck assembly further comprises guide rails, sliders, fixation bases and stop pins, the guide rails extending radially with respect to the base, the sliders slidably provided on the guide rails, the fixation bases fixed to the sliders, with each of the jaws being secured to a respective one of the fixation bases, the stop pins fixed to the fixation bases, wherein the turntable defines curved stop slots, the stop pins are movably inserted in the stop slots, and opposite ends of the stop slots are spaced from a center of the turntable at different distances:
as a result of the turntable rotating in the first direction, the stop pins move from proximal ends of the curved stop slots to distal ends thereof, thereby causing the jaws to move outwardly, and the stop pins stop upon reaching and buckling the distal ends of the stop slots; and
as a result of the turntable rotating in the second direction, the stop pins move from the distal ends of the curved stop slots to the proximal ends thereof, thereby causing the jaws to move inwardly, and the stop pins stop upon reaching and buckling the proximal ends of the stop slots.

9-11. (canceled)

12. The device for producing microneedles according to claim 1, further comprising a tray for carrying the microneedle female mold therein, wherein the tray is able to carry microneedle female mold of different sizes.

13. The device for producing microneedles according to claim 12, further comprising a loading conveyor, an unloading conveyor and a transfer conveyor, the loading conveyor disposed in a loading zone, the unloading conveyor disposed in an unloading zone,
the loading conveyor configured to transport the tray into the feeding cavity, the unloading conveyor configured to receive the tray from the discharging cavity,
the transfer conveyor configured to receive the empty tray from the unloading conveyor and transport the empty tray to the loading zone.

14. The device for producing microneedles according to claim 13, wherein the transfer conveyor is disposed under the loading conveyor and the unloading conveyor, wherein the device further comprises an automated transport device for transporting the empty tray from the unloading conveyor to the transfer conveyor and transporting the empty tray from the transfer conveyor to the loading conveyor.

15. The device for producing microneedles according to claim 13, further comprising an automated feeding mechanism and an automated loading mechanism both disposed in the loading zone, the automated feeding mechanism configured to transport the microneedle female mold to a loading location,
the automated loading mechanism configured to pick up the microneedle female mold from the loading location and place the microneedle female mold into the tray on the loading conveyor, and/or
the device for producing microneedles further comprising an automated unloading mechanism and a transfer tray both disposed in the unloading zone, the automated unloading mechanism configured to pick up the microneedle female mold from the tray on the unloading conveyor and place the microneedle female mold into the transfer tray.

16-19. (canceled)

20. A method for producing microneedles, comprising:
providing a microneedle female mold, a surface of the microneedle female mold provided with grooves matching with the microneedles;
placing the microneedle female mold into a feeding cavity in a non-negative pressure state and then drawing a vacuum in the feeding cavity by an evacuation mechanism so that the feeding cavity and the microneedle female mold are both in a negative pressure state;
transporting the microneedle female mold in the negative pressure state from the feeding cavity in the negative pressure state into a filling cavity in the negative pressure state, casting a solution for producing the microneedles, in the negative pressure state, onto the surface of the microneedle female mold by a filling mechanism and performing a leveling process on the microneedle female mold with the solution cast thereon by a leveling mechanism so that the solution is uniformly flattened on the surface of the microneedle female mold;
transporting the microneedle female mold with the solution casted thereon from the filling cavity in the negative pressure state into a discharging cavity in the negative pressure state and maintaining the filling cavity in the negative pressure state;
after the microneedle female mold with the solution casted thereon is transported into the discharging cavity in the negative pressure state, destroying a vacuum in the discharging cavity in the negative pressure state so that the discharging cavity is in the non-negative pressure state, and obtaining the microneedle female mold with the solution thereon; and
after the microneedle female mold with the solution thereon solidifies and is molded, performing a demolding process, thereby obtaining the microneedles.

21. The method for producing microneedles according to claim 20, further comprising:
after the microneedle female mold in the negative pressure state is transported from the feeding cavity in the negative pressure state into the filling cavity in the negative pressure state, destroying a vacuum in the feeding cavity in the negative pressure state so that the feeding cavity is in the non-negative pressure state and gets ready for reception of the next microneedle female mold therein; and/or
the method for producing microneedles further comprising:
after the vacuum in the discharging cavity in the negative pressure state is destroyed, transporting the microneedle female mold with the solution thereon out of the discharging cavity in the non-negative pressure state, thereby emptying the discharging cavity in the non-negative pressure state; and
after the discharging cavity in the non-negative pressure state is emptied, drawing a vacuum in the discharging cavity by the evacuation mechanism so that the discharging cavity is in the negative pressure state and ready for reception of the next microneedle female mold that has been undergone a leveling process.

22. (canceled)

23. The method for producing microneedles according to claim 20, further comprising:
at a casting location in the filling cavity, casting the solution onto the surface of the microneedle female mold in the negative pressure state by the filling mechanism; and
at a leveling location in the filling cavity, performing the leveling process on the microneedle female mold with the solution cast thereon by the leveling mechanism,
wherein at the leveling location, the microneedle female mold is carried in a tray, wherein the leveling mechanism drives the tray to move and thereby effectuates the leveling process on the microneedle female mold, the movement of the tray comprising at least one of horizontal rotation, horizontal translation, shaking and vertical swinging.

24. (canceled)

25. The method for producing microneedles according to claim 20, comprising: passing the microneedle female mold in a tray successively through the feeding cavity, the filling cavity and the discharging cavity, wherein microneedle female mold of different sizes can be transported in the tray: transporting the tray into the feeding cavity on a loading conveyor in the loading zone and receiving the tray from the discharging cavity onto an unloading conveyor in an unloading zone; and
receiving the empty tray from the unloading conveyor onto a transfer conveyor and transporting the empty tray on the transfer conveyor into the loading zone.

26. (canceled)

27. The method for producing microneedles according to claim 25, wherein the transfer conveyor is arranged under the loading conveyor and the unloading conveyor, wherein the method further comprises:
transporting the empty tray from the unloading conveyor onto the transfer conveyor by an automated transport device and transporting the empty tray from the transfer conveyor onto the loading conveyor by the automated transport device; and/or
the method for producing microneedles further comprising:
transporting the microneedle female mold to a loading location by an automated feeding mechanism in the loading zone, and
picking up the microneedle female mold from the loading location and placing the microneedle female mold into the tray on the loading conveyor by an automated loading mechanism in the loading zone; and/or
the method for producing microneedles further comprising:
picking up the microneedle female mold from the tray on the unloading conveyor and placing the microneedle female mold into the transfer tray in the unloading zone by an automated unloading mechanism in the unloading zone.

28-31. (canceled)

32. A method for producing microneedles, comprising:
providing a microneedle female mold and a vacuum chamber, a surface of the microneedle female mold provided with grooves matching with the microneedles, the vacuum chamber comprising a filling cavity and a discharging cavity, which are independent of each other;
placing the microneedle female mold into the filling cavity in a non-negative pressure state and casting a solution for producing the microneedles onto the surface of the microneedle female mold by a filling mechanism;
performing a leveling process on the microneedle female mold by a leveling mechanism so that the solution is uniformly flattened on the surface of the microneedle female mold;
drawing a vacuum in the filling cavity by an evacuation mechanism so that the filling cavity and the microneedle female mold are both in a negative pressure state;
transporting the microneedle female mold having undergone the leveling process into the discharging cavity in the negative pressure state, destroying a vacuum in the discharging cavity in the negative pressure state so that the discharging cavity is in the non-negative pressure state, and obtaining the microneedle female mold with the solution thereon; and
after the microneedle female mold with the solution thereon solidifies and is molded, performing a demolding process.

33. The method for producing microneedles according to claim 32, further comprising:
at a casting location in the filling cavity, casting the solution onto the surface of the microneedle female mold by the filling mechanism; and
at a leveling location in the filling cavity, performing the leveling process on the microneedle female mold with the solution cast thereon by the leveling mechanism.

34. The method for producing microneedles according to claim 33, wherein the microneedle female mold is passed through the filling cavity and the discharging cavity in a tray, wherein at the leveling location, the leveling mechanism drives the tray to move and thereby effectuates the leveling process on the microneedle female mold, the movement of the tray comprising at least one of horizontal rotation, horizontal translation, shaking and vertical swinging.
